(19) United States
(12) Patent Application Publication
(10) Pub. No.: US 2004/0265927 A1
(43) Pub. Date: Dec. 30, 2004

(54) Apoptosis-mimicking synthetic entities and use thereof in medical treatment

(76) Inventors: Anthony E. Bolton, Dublin (GB); Arkady Mandel, Ontario (CA); Stephen J. Houldsworth, Ontario (CA); Natalie J. Lazarowych, Ontario (CA)

Correspondence Address: FOLEY & LARDNER LLP, THREE PALO ALTO SQUARE, 3000 EL CAMINO REAL, SUITE 100, PALO ALTO, CA, 94306, US

(21) Appl. No.: 10/488,214

(22) PCT Filed: Sep. 13, 2002
(86) PCT No.: PCT/CA02/01398

Related U.S. Application Data
(60) Provisional application No. 60/323,234, filed on Sep. 18, 2001.

(51) Int. Cl. G01N 33/574; C12N 9/64

(52) U.S. Cl. 435/7.23; 435/226; 525/54.1

(57) ABSTRACT

Synthetic bodies having a thee-dimensional structure, sized and shaped to resemble apoptotic cells and apoptotic bodies, and comprising phospho-amino acid-side group carrying entities such as beads, are provided. They can be administered to a patient, to alleviate a variety of disorders such as T-cell mediated disorders (autoimmune conditions), inflammatory disorders neurodegenerative disorders and endothelial dysfunction disorders.

### FIELD OF THE INVENTION

This invention relates to novel chemical entities and compositions thereof, having biochemical activity, and to the uses thereof in the treatment and/or prophylaxis of various disorders in mammalian patients. More particularly, it relates to novel synthetic bodies which can mimic natural apoptotic bodies and cells after introduction into the body of a patient, to produce beneficial effects, and to their preparation and use.

### REFERENCES

The following documents are cited herein:

1. Kerr, J. F. R., Wyllie A. H., Currie, A. R. (1992), “Apoptosis: a basic biological phenomenon with wide-ranging implications in tissue kinetics.” British Journal of Cancer 26: 239-257;

2. Fadok, V. A. et. al., (1998), “Macro[phages that have ingested apoptotic cells in vitro inhibit proinflammatory cytokine production through autocrine/paracrine mechanisms involving TGF-beta, PGE2 and PAF,” J. Clin. Invest, 101, 890-898;

3. Fadok V. A., Voelker D. R., Campbell P. A., Cohen, J. J., Bratton, D. L., Henson, P. M. (1992), “Exposure of phosphatidylserine on the surface of apoptotic lymphocytes triggers specific recognition and removal by macrophages.” Journal of Immunology, 148:2207-2216;

4. Fadok V. A., Bratton, D. L., Rose, D. M., Pearson, A., Exekewitz R. A. B., Henson, P. M. (2000), “A receptor for phosphatidylserine-specific clearance of apoptotic cells,” Nature 405:85-90;

5. Monastra et al. Neurology (1993) 48:153-163;

6. Griffin W S T, Stanley, L. C., Ling, C., White, L., Macleod, V. Perrot L. H J., White, C. L., Araoz, C., (1989), Brain interleukin 1 and S-100 immunoreactivity are elevated in Down's syndrome and Alzheimer's disease, Proceedings of the National Academy of Sciences USA 86: 7611-7615;

7. Mogi M., Harada, M., Narabayashi, H., Inagaki, H., Minami, M., Nagatsu T., (1996) “Interleukin (IL)-1 beta, IL-1, IL-4, IL-6 and transforming growth factor-alpha levels are elevated in ventricular cerebrospinal fluid in juvenile parkinsonism and Parkinson's disease,” Neuroscience Letters 211: 13-16;

8. Murray, C. A., Lynch, M. A., (1998) “Evidence that increase hippocampal expression of the cytokine interleukin-1β is a common trigger for age and tress-induced impairments in long-term potentiation,” Journal of Neuroscience 18:2974-2981;

9. Bliss, T. V. P., Collinridge, G. L., (1993) “A synaptic model of memory: long-term potentiation in the hippocampus,” Nature 361:31-39.

All of the above references are herein incorporated by reference in their entirety to the same extent as if each individual reference was specifically and individually indicated to be incorporated herein by reference in its entirety.

State of the Art

Two mechanisms of call death in the body are recognized, necrosis and apoptosis. Apoptosis is the process of programmed cell death, described by Kerr, et al. (1992) by which steady-state levels of the various organ systems and tissues in the body are maintained as continuous cell division is balanced by cell death. Cells undergoing apoptosis often exhibit distinctive morphological changes such as pronounced decrease in cell volume, modification of the cytoskeletons resulting in pronounced membrane blebbing, a condensation of the chromatin, and degradation of the DNA into oligonucleosomal fragments. Following these morphological changes, an apoptotic cell may break up into a number of small fragments known as apoptotic bodies, consisting essentially of membrane-bound bodies containing intact organelles, chromatin, etc. Apoptotic cells and apoptotic bodies are normally rapidly removed from the body by phagocytosis principally by macrophages and dendritic cells, before they can become lysed and release their potentially pro-inflammatory intracellular contents.

Macrophages which have ingested apoptotic cells and/or apoptotic bodies appear to inhibit pro-inflammatory cytokine production (Fadok et al., 1998) and thus may down-regulate a Th-1 response in a patient's immune system following injection of apoptotic cells or bodies, or following injection of cells susceptible to accelerated apoptosis, upon phagocytosis thereof.

During apoptosis, phosphatidylserine becomes exposed externally on the cell membrane (Fadok V. A. et al. (1992)), and this exposed phosphatidylserine binds to specific receptors to mediate the uptake and clearance of apoptotic cells in mammals (Fadok V. A. et al. (2000)). The surface expression of phosphatidylserine on cells is a recognized method of identification of apoptotic cells.

Monastra et al. (1993) describe that the administration of phospholipid phosphatidylserine (PS) derived from bovine cortex (BC-PS), in extremely high dose, 30 mg/kg, may have an effect on adoptively transferred Experimental Autoimmune Encephalomyelitis (EAE) in SJL/J mice.

### SUMMARY OF THE INVENTION

The present invention provides synthetic chemical entities which, upon administration to a mammalian patient, will mimic apoptotic cells and/or bodies with consequent down-regulation of pro-inflammatory cytokines and/or upregulation of anti-inflammatory cytokines. The chemical entities comprise biocompatible derivatized bodies such as beads of a size similar to that of a mammalian apoptotic cell or apoptotic body derived from an apoptotic cell, the beads having, exposed on their surfaces, phospho-amino acid groups which will interact with PS or other appropriate receptors on antigen presenting cells in the patient=s body. These groups are appropriately spaced from the bodies so that the antigen presenting cells can engulf the bodies as the phospho-amino acid groups interact with the receptors in an in vivo process resembling the uptake of natural apoptotic cells or bodies, with consequent down-regulation of pro-inflammatory cytokines and/or upregulation of anti-inflammatory cytokines. Consequently, the chemical entities can be used for therapeutic purposes, for treatment and/or prophylaxis of a wide range of mammalian disorders in which pro-inflammatory or anti-inflammatory cytokines are implicated.

Thus according to a first aspect of the present invention, there are provided biocompatible synthetic entities comprising:

a three-dimensional head portion of size in its largest dimension of from 50 nanometers to 500 microns;

a plurality of tail portions bonded to each said head portion, the tail portions having:

phospho-amino acid end groups capable of interaction with receptors on antigen-presenting cells,

and chemical spacer groups of at least 3 linear carbon atoms,

the spacer groups being bonded at their proximal ends to the respective head portion, and at their distal ends to the phosphate of the phospho-amino acid group.

### BRIEF REFERENCE TO THE DRAWINGS

FIG. 1 is a reaction scheme showing the synthetic process of preparation of the preferred phosphoamino acid side chain of the invention;

FIG. 2 is a similar reaction scheme showing the synthetic process of coupling the side chains to the PMMA beads, Example 2;

FIG. 3 is a graphical presentation of the experimental results obtained in the specific Example 3 below, a measurement of ear swelling in a murine model of contact hypersensitivity in animals treated with the entities of the invention in comparison with control.

### DESCRIPTION OF THE INVENTION AND PREFERRED EMBODIMENTS

According to the present invention, synthetic entities which have the property of mimicking apoptotic cells and/or apoptotic bodies in that they are taken up by cells of the patient's immune system with accompanying beneficial effects such as inhibition of pro-inflammatory cytokines in vivo and/or promotion of anti-inflammatory cytokines in vivo are provided, and are administered to patients. These synthetic entities are three dimensional bodies having shapes and dimensions ranging from those resembling mammalian cells to shapes and dimensions approximating to apoptotic bodies produced by apoptosis of mammalian cells, and having phospho-amino acid such as phospho-serine groups attached to the surface thereof through intermediary chemical chains of appropriate length, and having the ability to interact with receptors such as PS receptors on antigen presenting cells of the mammalian body. Such bodies are hereinafter referred to as “phospho-amino acid-carrying beads.”

As noted above, exposed PS on the membrane of a cell is known to play a key role in the clearance of apoptotic lymphocytes by macrophages. A receptor for PS is present on macrophages. A “phosphatidylserine receptor” or “PS receptor” is a receptor on an antigen presenting cell (APC), such as a macrophage, whose activity is blocked by soluble phosphatidylserine, either monomeric or oligomeric. It is contemplated that the PS receptor may also be present on other APCs, such as dendritic cells and B cells.

According to this invention, phospho-amino acid-carrying beads interact with a patient's immune system, after administration to the patient by suitable means, presumably by engulfment by or other interaction with macrophages or dendritic cells or other antigen-presenting cells, to give substantially similar effects in terms of cytokine responses as are obtained when apoptotic cells/bodies are phagocytosed by macrophages. The phospho-amino acid groups with their chemical chain attachments to the beads play the role of phosphatidylserine in apoptotic cells, in a process mimicking phagocytotsis, with the beads playing the role of the apoptotic cell body.

Preferably, the phospho-amino acid groups forming the end groups of the entities of the invention have the general formula:

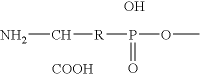

in which R represents C1-C4 straight chain or branched alkylene, alkylene-oxy, alkylene-thio, alkylene-amine, phenyl, iodo-substituted phenyl, and 5-membered N-heterocyclic groups, with the proviso that they interact with appropriate receptors on antigen-presenting cells.

Alternatively, it is contemplated that the phospho-amino acid groups forming the end groups of the entities of the invention can be attached to the spacer group through the carboxyl group or the amino group of the phosphoserine entity. In one embodiment, the carboxyl or amino group can be linked to the spacer via an amide group or a carbamate group. In the case of the carboxyl group, the linkage can alternatively be an ester group and in the case of the amino group, the linkage can alternatively be a urea group.

Preferred phospho-amino acid groups in the compositions of the present invention are phospho-serine and phosphothreonine groups, with the most preferred being phosphoserine of formula:

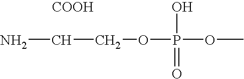

Preferably, the chemical spacer group by means of which the phospho-amino acid groups are linked to the head portion of the beads are suitably of length from 3-20 linear carbon atoms and 0-5 heteroatoms selected from oxygen, sulfur and >NR where R is hydrogen, alkyl of from 1-10 carbon atoms and phenyl, i.e., they contain chains of 3-20 carbon atoms and 0-5 heteroatoms linearly arranged between the head portion and the phospho group of the phospho-amino acid end group, irrespective of the presence of any branches or side chains on linear chain of the chemical spacer group. Typically the chemical spacer group is a linear ester group.

In a particularly preferred embodiment, the tail portions of the derivatized beads have the chemical formula:

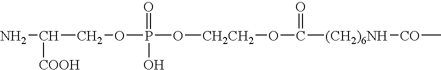

the amide end group being bonded to the head portion surface. In this example, the spacer comprises the group

—CH₂CH₂OC(O)—(CH₂)₆—NHC(O)—

which has 10 carbon atoms as well as 2 heteroatoms in the linear chain.

The term Abeads® as used herein is intended to mean substantially any biocompatible body, solid, semisolid or hollow, shape-retaining and typically but not exclusively spheroidal, cylindrical, ellipsoidal including oblate and prolate spheroidal, serpentine, reniform, etc., and from about 50 nanometers to about 500 microns in diameter. They may be flexible or rigid and soluble or insoluble in aqueous solutions such as blood. Preferred materials for their composition are polymethylmethacrylate, polyacrylate, polymethacrylate, glass, polystyrene, polyethylene, polypropylene and the like, of a grade approved for administration to mammalian patients.

Procedures for coupling the tail portions defined above to beads are well known in the art with suitable exemplification provided in the examples below.

The phospho-amino acid-carrying synthetic entities of the invention may be administered to the patient by any suitable means which brings them into operative contact with active components of the patient's immune system. Preferably, the entities are constituted into a liquid suspension in a biocompabble liquid such as physiological saline and administered to the patient intra-arterially, intravenously or most preferably intramuscularly or subcutaneously.

A preferred manner of administering the synthetic entities to the patient is as a course of injections, administered daily, several times per week, weekly or monthly to the patient, over a period ranging from a week to several months. The frequency and duration of the course of the administration is likely to vary widely from patient to patient, and according to the condition being treated, its severity, and whether the treatment is intended as prophylactic, therapeutic or curative. Its design and optimization is well within the skill of the attending physician.

The quantities of synthetic entities to be administered will vary quite widely depending on the nature of the mammalian disorder it is intended to treat and on the identity and characteristics of the patient. It is important that the effective amount of entities is non-toxic to the patient, and is not so large as to overwhelm the immune system. When using intra-arterial, intravenous, subcutaneous or intramuscular administration of a liquid suspension of entities, it is preferred to administer, for each dose, from about 0.1-50 ml of liquid, containing an amount of PS-carrying bodies generally equivalent to 1.0%-1000% of the number of cells normally found in an equivalent volume of whole blood or the number of apoptotic bodies that can be generated from them. Generally, the number of synthetic entities administered per delivery to a human patient is suitably in the range from about 500 to about 20,000,000.

Since the synthetic entities are acting, in the process-of the invention, as immune system modifiers, in the nature of a vaccine, the number of such bodies administered to an injection site for each administration is a more meaningful quantitation than the number or weight of synthetic entities per unit of patient body weight. For the same reason, effective amounts or numbers of synthetic entities for small animal use may not directly translate into effective amounts for larger mammals on a weight ratio basis. The amounts can thus be in the range 500-2×10⁹, and preferably within the range 10,000-2×10⁹ synthetic entities per injection.

While it is not intended that the scope of the present invention should be limited by any particular theories of its mode of operation, the following is offered as a tentative explanation, for a better understanding of the ways an means by which the invention may be put into practice. It is postulated that antigen-presenting cells of the patient's immune system, notably professional antigen-presenting cells (APCs) including macrophages and dendritic cells, take up the phosphato-amino acid-carrying synthetic entities in a similar manner to the way in which they would take up apoptotic cells and apoptotic bodies. Having taken up the entities, the APCs induce an anti-inflammatory response promoting a change in the Th cell population with an increase in the proportion of Th2 cells and/or other regulatory/anti-inflammatory cell populations (e.g., Th1 cells), and a decrease in Th1 cells. Th2 cells and other regulatory cells secrete anti-inflammatory cytokines such as interleukin-10, leading to reduced inflammation.

The present invention is indicated for use in prophylaxis and/or treatment of a wide variety of mammalian disorders where T-cell function, inflammation, endothelial dysfunction and inappropriate cytokine expression are involved. A patient having, suspected of having, or being particularly prone to contracting such a disorder may be selected for treatment. A Treatment@ means a reduction in symptoms, such as, but not limited to, a decrease in the severity or number of symptoms of the particular disorder.

In respect of T-cell function (T-cell mediated) disorders, these may be autoimmune disorders including but not limited to diabetes, scleroderma, psoriasis and rheumatoid arthritis. The invention is indicated for use with inflammatory allergic reactions, organ and cell transplantation reaction disorders, and microbial infections giving rise to inflammatory reactions. It is also indicated for use in prophylaxis against oxidative stress and/or ischemia-reperfusion injury, ingestion of poisons, exposure to toxic chemicals, radiation damage, and exposure to airborne and water-borne irritant substances, etc., which cause damaging inflammation. It is also indicated for inflammatory, allergic and T-cell-mediated disorders of internal organs such as kidney, liver, heart, etc.

With respect to disorders involving inappropriate cytokine expression for which the present invention is indicated, these include neurodegenerative diseases. Neurodegenerative diseases, including Down's syndrome, Alzheimer's disease and Parkinson's disease, are associated with increased levels of certain cytokines, including interleukin-1β (IL-1β) (see Griffin WST et al. (1989), and Mogi M. et al. (1996)). It has also been shown that II-1β inhibits long-term potentiation in the hippocampus (Murray, C. A., et al. (1998)). Long-term potentiation in the hippocampus is a form of synaptic plasticity and is generally considered to be an appropriate model for memory and learning (Bliss, T. V. P. et al. (1993)). Thus, inappropriate cytokine expression in the brain is currently believed to be involved in the development and progression of neurodegenerative diseases.

Thus, the invention is indicated for the treatment and prophylaxis of a wide variety of mammalian neurodegenerative and other neurological disorders, including Downs syndrome, Alzheimer's disease, Parkinson's disease, senile dementia, depression, multiple sclerosis, Huntingdon's disease, peripheral neuropathies, spinal cord diseases, neuropathic joint diseases, chronic inflammatory demyelinating disease, neuropathies including mononeuropathy, polyneuropathy, symmetrical distal sensory neuropathy, neuromuscular junction disorders, myasthenias and amyotrophic lateral sclerosis.

Regarding disorders involving endothelial dysfunction, the present invention is indicated for the treatment and prophylaxis of a wide variety of such mammalian disorders including, but not limited to, cardiovascular diseases, such as atherosclerosis, peripheral vascular disease, congestive heart failure, stroke, myocardial infarction, angina, hypertension, etc., vasospastic disorders such as Raynaud's disease, cardiac syndrome X, migraine etc., and the damage resulting from ischemia (ischemic injury or ischemia-reperfusion injury). In summary, it can be substantially any disorder that results from an inappropriately functioning endothelium.

Processes of preparation of the phospho-amino acid-carrying beads of the invention depend to a large extent on the nature of the starting material, i.e. the bead and its chemical composition. In the case of the preferred polymethylmethacrylate beads, these generally have surface reactive groups such as carboxylic acid groups which provide suitable sites of chemical attachment for the desired phospho-amino acid bearing side groups. In a first synthetic step, an amine terminated straight chain alkanol such as 8-amino-octan-1-ol, 7-amino-heptan-1-ol or 6-amino-hexan-1-ol is chemically protected at its amine terminus, and then reacted at its free hydroxy end group with an appropriate phospho compound, such as POCI3 to form a phospho-terminated compound capable of subsequent reaction with an amino acid to form the desired phospho-amino acid grouping. Then the amine protectant is chemically removed, and the compound is reacted with the carboxylic acid groups on the polymethylmethacrylate beads, to form an amide linkage thereto. Deprotection of the amine groups and reaction with the polymethylmethacrylate beads leads to formation of the phosphatido-amino acid-carrying beads of the present invention.

Examples of appropriate phosphate compounds for use in making phosphato-amino acid-carrying beads according to the present invention will be apparent to those skilled in the art.

Serine is the preferred amino acid for use in the phospho-amino acid-carrying beads, so as to provide an end phospho-amino acid group most capable of interaction with the phosphatidylserine receptors of the macrophages and other phagocytosing cells after administration to the patient. Other amino acids which will perform the same or substantially the same function may also be used.

The invention is further described for illustrative purposes in the following specific examples.

### EXAMPLE 1

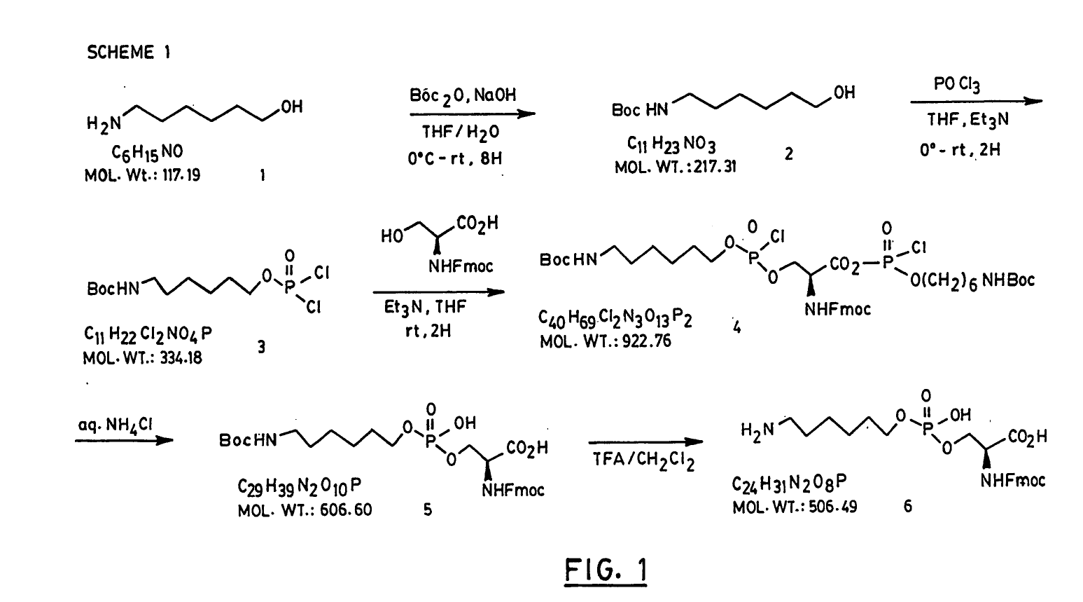

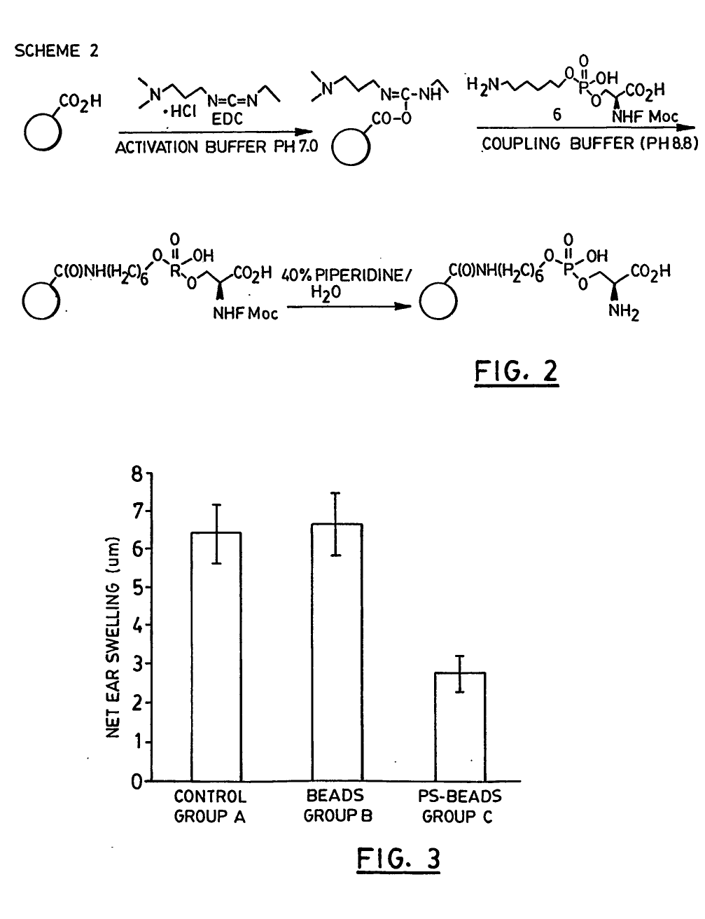

The chemical procedure of the preparation of the phosphoamino acid side chain group is illustrated diagrammatically in accompanying FIG. 1. The procedure of chemical attachment of the side chains to beads is diagrammatically illustrated in accompanying FIG. 2.

1. Preparation of 6-(N-tert-butyloxycarbonylamino)-1-hexanol 2

A three-necked 250 mL round bottom flask was fitted with a magnetic stirring bar, a 100-mL addition funnel, a thermometer and placed in an ice bath. The flask was charged with 6-amino-1-hexanol (5.0 g, 85.3 mmol, 1.0 eq.), DMF (25 ml) and an aqueous sodium hydroxide solution (2.1 g NaOH in 20 mL of water). After cooling to 0° C., a solution of di-tert-butyldicarbonate (11.2 g, 102 mmol, 1.2 eq.) in DMF (20 ml) was added dropwise to the solution, keeping internal temperature at 14 B 15° C. To the reaction mixture was added water (200 mL) and dichloromethane (200 mL). The layers were separated and the water layer was extracted with dichloromethane (4×50 mL). The combined organic layers were washed with water (4×75 mL) and dried over sodium sulphate. The solvent was then removed under vacuum to give 7.1 g of product 2 (FIG. 1) as a white solid (KD-745, yield: 76.3%). ¹H NMR (CDCl₃): δ4.54 (s, br, 1H, OH); 3.67 (t, 2H, CH₂O); 3.15 (s, br, 2H, CH₂N); 1.49 (s, 9H, t-Bu); 1.30 B 1.65 (m, 8H, 4CH₂).

2. Preparation of O-{6-(N-tert-butyloxycarbonylamino)-1-hexyl]phosphoryl}-N-fluorenylmethoxycarbonylamino-L-Serine 5

A three-necked 500-mL round bottom flask was fitted with a magnetic stirring bar, an argon inlet, a 250-mL addition funnel and placed in an ice bath. The flask was charged with phosphorus oxychloride (86 mL, 920 mmol, 100 eq.) and anhydrous THF (100 mL). After cooling to 0° C., a solution of 6-(N-tert-butyloxycarbonylamino)-1-hexanol 2 (2.0 g, 9.2 mmol, 1.0 eq.), triethylamine (1.4 mL, 9.2 mmol, 1.0 eq.) and anhydrous THF (100 mL) was added dropwise over 30 min. The mixture was stirred at 0° C. for 1 h and then at room temperature for 1 h. The reaction mixture was transferred to a one-necked 250-mL round bottom flask and evaporated at 30° C. under high vacuum to remove THF and excess of phosphorus oxychloride. After coevaporating with toluene (2×100 mL) at 30° C. under high vacuum, the residue was used directly in the next step.

The one-necked 250-mL round bottom flask containing the residue from the last step was treated with anhydrous THF (100 mL) and was fitted with a magnetic stirring bar and a septum seal. After cooling to 0° C., a solution of L-Fmoc-Ser (1.51 g, 4.6 mmol, 0.5 eq.) in THF (20 mL) and another solution of triethylamine (1.3 mL, 9.2 mmol, 1.0 eq.) in anhydrous THF (10 mL) were added dropwise through syringes simutaneously. After the addition the mixture was stirred at 0° C. for 1 h, and then at room temperature overnight. The reaction mixture was concentrated to dryness under vacuum. To the residue was added saturated aqueous ammonium chloride solution (100 mL) and a mixture of t-butanol and ethyl acetate (1:3, 100 mL). The aqueous layer was extracted with t-butanol and ethyl acetate (1:3) (4×50 mL). The organic layer was dried over sodium sulphate. After concentration in vacuo 5.0 g of crude product was obtained. TLC showed at lease 5 products TLC (silica gel, IPA/CH₂Cl₂/HOAc 1:5:1 drop). They were separated by preparative TLC using the same TLC solvent system. NMR and MS showed one of fractions was the target compound 5. (KD-7-59-2, R_{f-product}=0.29, R_{f-F-moc-Ser}=0.17, silica gel, eluent: IPA/CH₂Cl₂/HOAc 1:5:1 drop; yield: 0.3 g, 5.4%).

3. Preparation of O-[(6-amino-1-hexyl)phosphoryl]-N-fluorenylmethoxycarbonylamino-L-Serine 6

A one-necked 100-mL round bottom flask was fitted with a magnetic stirring bar, a septum seal, an argon inlet and placed in an ice bath. The flask was charged with 0.20 g of O-{6-(N-tert-butyloxycarbonylamino)-1-hexyl]phosphoryl}-N-fluorenylmethoxy-carbonylamino-L-Serine 5 (FIG. 1) and cooled to 0° C. A solution of trifluoroacetic acid (1.4 mL) in CH₂Cl₂ (5.5 mL) was added in portions. The mixture was stirred at room temperature for 30 min. TLC showed the reaction was incomplete (silica gel, IPA/H₂O/NH₄OH 10:2:1). To the mixture was added 1.4 mL more of TFA. After stirring for 40 min. TLC showed the reaction was complete. The reaction mixture was concentrated to dryness and coevaporated with ethyl acetate (3×10 mL). Drying under high vacuum gave 133 mg (GM-9-25-2) of the product 6(FIG. 1) which was used directly in the next step.

### EXAMPLE 2

### Coupling Reaction of O-[(6-amino-1-hexyl)phosphoryl]-N-fluorenylmethoxycarbonylamino-L-Serine 6 With Polybead⁷

The procedure of chemical attachment of the side chains to beads is diagrammatically illustrated in accompanying FIG. 2.

Reagents and Conditions

Polybead⁷, Poly(methyl methacrylate) or PMMA (5.15% solids-latex) was obtained from Polysciences Inc., 400 Vallet Roab, Warrington, Pa., U.S.A. 18976, Product Number 23570. The suspension contains PMMA and water, 100-200 μmol of carboxy per gram of polymer. 1.8×10¹³ particles per gram, d=1.19 g/ml, 11.0 mL, 11.64 g. 11.64 (g)×5.15%×200=120 μmol of carboxy, 1.0 eq. Activation Buffer (pH 7.0-7.5), sodium phosphate monobasic (0.22 g/l, MW 120.0), pH adjusted to 7.01 using 1N NaOH; Coupling Buffer (pH 8.6), sodium phosphate monobasic (0.22 g/l, MW 120.0), pH adjusted to 8.80 using 1N NaOH 1-Ethyl-(3-dimethylaminopropyl) carbodiimide hydrochloride (EDC), anhydrous, M.W. 191.7, mp 117° C., Chem-Impex Cat# 00050, lot# Y238T.

NH₂(CH₂)₆OP(O)(OH)(L-Fmoc-Ser-OH) or NH₂C₆H₁₂O-P-Fmoc-Ser-OH or NH₂—HPS—OH (0.110 g, 217 μmol, C₂₄H₃₁N₂O₈P, Mol. Wt.: 506.49, 1.8 eq.) from our synthesis. Keep the centrifuge tubes capped tightly always during centrifuging.

Coupling PMMA With NH₂(CH₂)₆OP(O)(OH)(L-Fmoc-Ser-OH) in Water

Three centrifuge tubes (MILIPORE, CENTRICON PLUS-20 with centrifugal filters) were tare weighed and the PMMA suspension was transferred to the centrifuge tubes: #1 (2.91 g), #2 (2.91 g) and #3 (5.82 g). Total: 11.0 mL, 11.64 g, 11.64 (g)×5.15%×200=120 μmol, 1.0 eq. They were centrifuged for 15 min. at 12700 rpm to separate the supernatant.

The activation buffer was added to the centrifuge tubes: #1 (2.5 mL), #2 (2.5 mL) and #3 (5.0 mL). The tubes were vortexed to redisperse. After centrifuging for 15 min. at 12700 rpm the clear supernatant was separated. Remove and separate supernatant. This step was repeated once.

The activation buffer was added to the centrifuge tubes: #1 (2.5 mL), #2 (2.5 mL) and #3 (5.0 mL). The tubes were vortexed to redisperse. A solution of EDC (2.3 g, FW. 191.7, 12000 mmol, 100 eq.) in the activation buffer (14.0 mL) was transferred to the centrifuge tubes: #1 (3.5 mL), #2 (3.5 mL) and #3 (7.0 mL). The tubes were shaken for 90 min. at room temperature and were centrifuged for 15 min. at 12700 rpm to separate the supernatant.

To the tubes was added the activation buffer. #1 (3.5 mL), #2 (3.5 mL) and #3 (7.0 mL). The tubes were vortexed to redisperse. The tubes were were centrifuged for 15 min. at 12700 rpm to separate the supernatant. This step was repeated twice.

NH₂—HPS—OH (0.110 g, 217 μmol, C₂₄H₃₁N₂O₈P, Mol. Wt.: 506.49, 1.8 eq.) was dissolved in 8.0 mL of coupling buffer and 3.0 mL of DMF, adjusted pH to 8.75 using 0.5 N NaOH solution to give 14.0 mL of a solution. To the tubes was added the solution: #1 (3.5 mL), #2 (3.5 mL) and #3 (7.0 mL). The tubes were vortexed to redisperse. The mixture in the tubes was allowed to react at room temperature for 61.5 hrs (19:00, 09/01/01 B 8:30, 01/04/01) with shaking.

The reaction mixture was centrifuged for 15 min. at 12700 rpm to separated the supernatant. The solid was collected from the centrifugal filter to give crude product A. The supernatant was collected and concentrated to dryness under high vacuum at 40° C. to give crude product B. Solids A and B were combined and transferred to a 20-mL centrifugal tube without filter.

HPLC grade water (8.0 mL) was added. The tubes were vortexed to redisperse. The reaction mixture was centrifuged for 15 min. at 12700 rpm to separate the supernatant. This step was repeated 3 times. The solid (0.55 g) was divided into two equal parts and put into two centrifugal tubes.

Deprotection With Aqueous Piperidine Soution

To one centrifugal tube was added a solution of piperidine (4.0 mL) in diethyl ether (6.0 mL). The mixture was shaken for 20 min. with vortexing for 1 min. every 5 min. It was found that a big piece of insoluble rubber-like gum was formed. To another centrifugal tube was added a solution of piperidine in water (2.0 mL of piperidine plus 3.0 mL of water). The mixture was shaken for 30 min. with vortexing for 1 min. every 5 min. The reaction mixture was centrifuged for 15 min. at 12700 rpm and the clear supernatant was separated.

HPLC grade water (6.0 mL) was added and the mixture was vortexed to redisperse. The reaction mixture was centrifuged for 15 min. at 12700 rpm to separate the supernatant. This step was repeated twice.

To the residual solid (0.15 g, wet) was added 10.0 mL of HPLC grade water filtered through 0.2 micron syringe filter. The suspension was vortexed for 20 min. and sonicated for 1 h to give the final suspension (GM-9-29).

The product was tested for amine by Kaiser test. [2 drops of ninhydrin solution in ethanol, 2 drops of phenol solution in ethanol, 2 drops of (2 mL 0.001 M aq. KCN solution+98 mL of pyridine), heat in the oven at 120° C. for 5 min.]. Results: dark blue, positive.

The final product name: O-(6-Polybead7aminohexyl)phosphoryl-L-Serine or Polybead7-C(O)NH(CH₂)₆OP(OH)(L-Ser-OH).

Specifications: Approximately 0.15 g of coupled Polybead7 in 10.0 mL of suspension (1.5 w/w %); Approximately 2.7×1011 beads per mL suspension; bead size 80-90 nanometers.

Loading Analysis of the Final Product

Two small test tubes A and B were labelled. To the test tube A was added 0.5 mL of final suspension (GM-9-29). The solvent was removed by freezing with dry-ice/1-PA and lyophilizing to give 4.0 mg of dry beads. The test tube B was a blank.

To both test tubes was added 50 μL of a phenol solution (40 g in 10 mL of abs. EtOH), 50 μL of a KCN solution (1.3 mg in 100 mL of pyridine), 25 μL of a ninhydrin solution (2.5 g in 50 mL of abs. EtOH). Both tubes were heated at 100° C. for 10 min.

Both samples A and B were diluted with 2.0 mL of 60% ethanol and transferred into Pasteur pipette containing a tight plug of glass wool. The residue of sample A was rinsed twice with 0.5 mL of 0.5 M Bu₄NHSO₄ solution (170 mg in 1.0 mL of water). Both the solutions were made up to 25.0 mL with 60% ethanol.

The absorbance of the solution A was measured at 570 nm against the blank solution B using UV-VIS spectrophotometer HP8452A). Sample absorbance: 0.348 (1.0 cm pathway).

Loading calculation (AI-3-27): μmol/g=(A₅₇₀$Vₘₗ/e₅₇₀$W_{mg})$10⁶=(0.348×25/1.5×10⁴×4.0)×10⁶=145 μmol/g

### EXAMPLE 3

### Utility

This example shows the effect of injecting phospho-amino acid-carrying beads of the present invention on ear swelling in the murine contact hypersensitivity (CHS) model:

Female BALB/c mice, age 6-8 weeks, weighing 22-25 g were obtained from Jackson Laboratories.

Phospho-amino acid-carrying beads were prepared as described in Examples 1 and 2 which had a phosphoserine concentration of 145 μmols/gm, bead size 80-90 nanometers.

Fourteen mice were assigned to group A, control, and no injections. Twelve mice were assigned to group B control, and received injections of suspensions of plain polymethylmethacrylate beads, 80-90 nanometer size, carrying no side chain derivatization. Another twelve mice were assigned to group C and received injections of the phosphoserine-carrying beads. Both groups B and C received the same volume injections and the same numbers of beads per injection.

The experiments were carried out over 7 days. Sensitization took place on day 1. For sensitization purposes, mice of groups B and C received their bead injections for day 1, and were anesthetized using 0.2 ml intraperitoneal (IP) injection of 5 mg/ml pentobarbital sodium. The abdominal skin of the mouse was sprayed with 70% ETOH. A blade was used to remove about a one-inch diameter of hair from the abdomen. The bare area was painted with 25 μl of 0.5% 2,4-dinitrofluorobenzene (DNFB) in 4:1 acetone:olive oil using a pipette tip. Control mice of group A were similarly sensitized, on the same day.

On each of days 1-6, experimental mice were injected with the phospho-amino acid-carrying beads Beads were suspended in physiological saline and injected in 50 μl volume containing 600,000 beads, via intramuscular (IM) injection. On Day 6, following bead injection for that day, mice were challenged with DNFB as follows: 10 μl of 0.2% DNFB was painted on the dorsal surface of the right ear with a pipette tip and 10 μl of vehicle was painted on the left ear with a pipette tip.

On Day 7, 24 hours after challenge, ear thickness was measured using a Peacock spring loaded micrometer, the animals being locally anesthetized with Halothane. Increase in ear swelling is used as a measure of CHS response. Data is expressed as the difference in the treated right ear thickness minus the thickness of the vehicle treated left ear, in microns. The significance of difference between the two experimental groups is determined by the two-tailed student t test. A value of p<0.05 is considered significant.

The results were as follows:

| Group | A | B | C |
|---|---|---|---|
| Average swelling | 6.43 | 6.67 | 2.75 |
| Std Dev | 2.87 | 2.84 | 1.54 |
| Std Err | 0.77 | 0.82 | 0.45 |
| % of control | 100 | 104 | 43 |
| % reduction | 0 | −4 | 57 |
| p value | | | 0.00058 |

The results are also presented graphically on FIG. 3 as a measurement of ear swelling in microns. Experimental group C show statistically significant improvement over control group A which received no injections, and the experimental group B which received injections of plain, underivatized beads.

All publications, patents and patent applications previously cited above are herein incorporated by reference in their entirety.

What is claimed is:

1. Biocompatible synthetic entities comprising:
a three-dimensional head portion of size in its largest dimension of from 50 nanometers to 500 microns;
a plurality of tail portions bonded to each said head portion, the tail portions having:
phospho-amino acid end groups capable of interaction with receptors on antigen-presenting cells,
and chemical spacer groups of at least 3 linear carbon atoms,
the spacer groups being bonded at their proximal ends to the respective head portion, and at their distal ends to the phosphate of the phospho-amino acid group.

2. Entities according to claim 1 wherein the phospho-amino acid groups forming the end groups of the entities of the invention have the general formula: 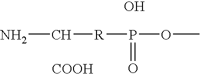
in which R represents C1-C4 straight chain or branched alkylene, alkylene-oxy, alkylene-thio, alkylene-amine, phenyl, iodo-substituted phenyl, and 5-membered N-heterocyclic groups.

3. Entities according to claim 2 wherein the phospho-amino acid groups are phospho-serine or phospho-threonine groups.

4. Entities according to claim 4 wherein the phospho-amino acid groups are phospho-serine groups of formula:

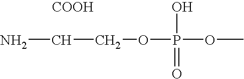

5. Entities according to any preceding claim wherein the head portions are beads of polymethylmethacrylate, polacrylate, polymethacrylate, glass, polystyrene, polyethylene, polypropylene or the like, of a grade approved for administration to mammalian patients.

6. Entities according to claim 5 wherein the head portions are beads of polymethylmethacrylate.

7. A process for alleviating the symptoms of a disorder in a mammalian patient, which comprises administering to the patient an effective amount of phospho-amino acid-carrying entitles as defined in claim 1.

8. The process of claim 7 wherein the disorder is a T-cell mediated disorder, an inflammatory disorder, an endothelial dysfunction disorder or an inappropriate cytokine expression disorder.

9. The process of claim 7 or claim 8 wherein the administration is conducted intra-arterially, intravenously, intramuscularly or subcutaneously, as a liquid suspension of phospho-amino acid-carrying entities in a biocompatible liquid.

10. The use in preparation of a medicament for alleviating the symptoms of a disorder in a mammalian patient, of phospho-amino acid-carrying entities as defined herein.

11. The use in alleviating the symptoms of a disorder in a mammalian patient, of phospho-amino acid-carrying entities as defined herein.

12. A method for treating a neurodegenerative disease comprising administering to a human a non-toxic effective neurodegenerative disease-treating amount of phospho-amino acid-carrying entities as defined herein.

13. A method for treating an immune system disorder characterized by an inappropriate cytokine expression, comprising administering to a human a non-toxic effective inappropriate-cytokine-expression-treating amount of phospho-amino acid-carrying entities as defined herein.

14. A method for treating an endothelial function disorder comprising administering to a human a non-toxic effective endothelial function disorder-treating amount of phospho-amino acid-carrying entities as defined herein.

15. A pharmaceutical composition comprising biocompatible synthetic bodies for administration to a mammalian patient and a pharmaceutically acceptable carrier wherein the biocompatible synthetic bodies comprise phospho-amino acid carrying entities according to any of claims 1-6.
